(19) United States
(12) Patent Application Publication
(10) Pub. No.: US 2004/0014894 A1
(43) Pub. Date: Jan. 22, 2004

(54) Epoxidized trialkylammonium salt, synthesis method and uses

(76) Inventors: Jacques Lemaire, Villeurbanne (FR); Christel Gozzi, Lyon (FR); Gilbert Boulon, Lyon (FR); Andre Dalphinet, Poleymieux Au Mont D'or (FR)

Correspondence Address: Oliff & Berridge, P O Box 19928, Alexandria, VA, 22320, US

(21) Appl. No.: 10/257,534

(22) PCT Filed: Apr. 19, 2001
(86) PCT No.: PCT/FR01/01214

(30) Foreign Application Priority Data
Apr. 19, 2000 (FR) 00/05059

(51) Int. Cl. C08L 35/00; C07D 303/08

(52) U.S. Cl. 525/217; 549/551

(57) ABSTRACT

The invention concerns an epoxidized trialkylammonium salt characterised in that it is selected among salts of general formula (I), wherein: R represents a (CH₂)ₙ—A—NH—(CH₂)_{n′}—N⁺R₁R₂R₃ group wherein A represents a —CO— or CH₂— group; n′ and n range between 1 and 20, with n+n′ not less than 10; R₁, R₂ and R₃ are C₁-C₂₀ alkyl or mono-hydroxyalkyl groups, linear or branched, identical or different; X represents counterion. The invention also concerns a synthesis method for said salts, the use of said salts as chemical grafting agents, a chemical grafting method for substrates and the resulting biocidal and/or bacteriostatic substrates.

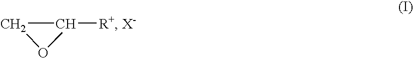

The present invention relates to the permanent modification of the properties, in particular with respect to microorganisms, of substrates, by covalent grafting of compounds having the properties sought for these substrates.

The properties sought for their benefit in the medical field, or in industrial sectors using cellulose in the form of paper, packaging and textile for example, are in particular the contact biocidal and/or bacteriostatic properties.

For all usable objects, for example in a sterile chamber, or by contrast in a contaminating medium, such as the components of trolleys used in the hospital sector, but also for example door handles and all objects which can be contaminated by contact and thus become contaminating, these contact biocidal and/or bacteriostatic properties may be of great interest.

For the industrialists of the adhesives or paints sector, or the manufacturers of cosmetic products for example, biocidal and/or bacteriostatic properties will be sought for starch and cellulose matrices.

The expression bacteriostatic is understood to mean the property which consists in inhibiting the development of microorganisms and in particular bacteria.

More precisely, the substrates exhibiting this contact bacteriostatic property are capable of inhibiting the development of microorganisms by mere contact. Those intrinsically exhibiting this property, such as modified starch and modified cellulose matrices, make it possible to inhibit the development of microorganisms coming into contact with them, for example by accidental contamination, without resorting to the addition of “preservatives”.

The expression biocide is understood to mean the property which consists in destroying a microorganism such as fungi, yeasts or bacteria.

More precisely, the substrates having this contact biocidal property are capable of causing the death of a microorganism by mere contact. Those intrinsically exhibiting this property, such as modified starch and modified cellulose matrices, make it possible to bring about the death of all microorganisms capable of contaminating them, without resorting to specific additives such as preservatives and disinfectants.

The methods used for modifying the substrates and conferring the desired particular properties on them should not damage the properties specific to these substrates; for example, when these substrates are textile fibers, their absorbent properties, when these substrates are cellulose preparations intended to be used in the form of packaging films, their mechanical resistance, and when starch intended for the preparation of adhesive or paint is involved, its thickening properties.

For the objects, if they are not protected by coating with a coat of paint or by a covering having the desired characteristics, grafting should not modify the physical properties of the mass, for example hardness, mechanical resistance and/or both physical and aesthetic surface properties.

The protection of substrates, in particular textiles or packagings, against agents such as bacteria, fungi or yeasts, by treating said substrates with compounds or products exhibiting a specific activity toward these microorganisms, is already well described.

These techniques which have already been described and used involve methods of treatment which may be divided into two categories:

on the one hand, the methods using means of ennobling by dipping, coating or any other means which will be assimilable to an impregnation, imbibition or deposition followed by simple drying, that is to say without modification of the chemical structure of the constituents, or creation of covalent chemical bonds,

and, on the other hand, the methods using means of ennobling by grafting of active compounds, by modification of the chemical structure of the substrates and creation of chemical bonds between the substrate and the compound having the desired properties.

The so-called impregnation techniques are used by adding products having the desired properties, for example to the last rinsing bath.

These techniques may have three major disadvantages due to the temporary character of the impregnation performed and the possible release of ennobling products:

the need to repeat the ennobling after prolonged use or after washing,

a risk of poor penetration of the ennobling products,

potential risks of intolerance and allergy in certain subjects.

The covalent grafting techniques make it possible to obtain products not exhibiting the abovementioned disadvantages, but the methods used or the products used are not economically viable or competitive with respect to the impregnation techniques.

There is known from document FR-A-2 511 001 the use of a grafting method, by treating polyhydroxylated or polyaminated polymers with salts of n-oxiranemethane N,N,N-trialkylaminium of formula:

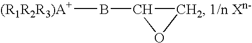

in which:

A represents a nitrogen or phosphorus atom,

R₁ and R₂ are chosen from the group consisting of C₁ to C₂₀ alkyl radicals or from the group consisting of C₁ to C₃ alkenyl radicals and aryl radicals which are optionally substituted,

it being possible for R₁ and R₂ optionally to form a heterocycle with A, when A is nitrogen,

R₃ is chosen from the group consisting of C1 to C20 alkyl radicals, C1 to C20 alkenyl radicals, aryl radicals which are optionally substituted and the radical

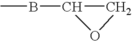

B is an alkylene radical between C₁ and C₁₀

Xⁿ⁻ is an anion,

n is a number equal to 1, 2 or 3.

In this document, the method for synthesizing the salts of N-oxiranemethane N,N,N-trialkylaminium has a relatively low yield of the order of 60 to 70%.

The compounds described in the French document FR-A-2 511 001 are, for example, N-oxiranemethanes, and no textile treated with these compounds currently exists on the market, to the best knowledge of the Applicant.

The solution developed was, however, already a response to the problems resulting from the use of the impregnation techniques.

According to the document FR-A-2 695 800, a method for covalent grafting, by free-radical activation under the effect of ionizing radiation such as gamma radiation, has been described.

This solution, by free-radical activation, using gamma radiation, of an ethylene functional group, as a replacement for the epoxide group, is motivated in particular by the difficulty of controlling a reaction using compounds containing epoxide groups, as described in the document FR-A-FR 2 695 800 cited above, and by the low yield of the method for synthesizing the reagents used.

However, the manufacture, on an industrial scale, of a biocidal textile by the method as described, and more particularly the step of grafting the biocidal compound onto the substrate with ionizing radiation, such as gamma radiation, represents a very heavy technological investment, and is not economically viable.

According to the document FR-A-2 781 489, the abovementioned disadvantages of the prior art were overcome by using a method of coating, without covalent grafting, with a copolymer of acrylamide or of acrylamide derivatives and trialkylammonium salts known for their biocidal activity.

The economic imperatives are satisfied by the use of these compounds, but the biocidal or bactericidal character of the substrates thus treated does not have the permanent character sought.

It is known that some trialkylammonium salts constitute a class of compounds possessing cleaning and disinfectant properties.

The Applicant has discovered a family of novel biocidal or bacteriostatic trialkylammonium salts containing an epoxide functional group allowing grafting by covalent bonding onto the substrate by opening of the epoxide functional group in a basic medium.

These novel trialkylammonium salts containing an epoxide functional group are easily accessible with very good yields, by the action of oxidants of the peracid or peroxide type, and preserve their biocidal properties after grafting.

A compound of the invention consists in a trialkylammonium salt of general formula (I):

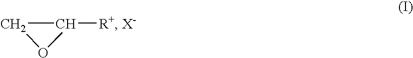

in which:

R represents a group:

—(CH₂)ₙ—A—NH—(CH₂)_{n′}—N⁺R₁R₂R₃

in which:

A represents a group —CO— or —CH₂—

n′ and n are each between 1 and 20, with (n+n′) greater than or equal to 10,

R₁, R₂ and R₃ are linear or branched C1 to C20 alkyl or monohydroxyalkyl groups, which may be identical or different,

X⁻ represents the counter-ion.

A preferred compound according to the invention is the compound of formula (II)

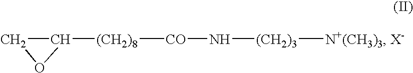

Another subject of the invention is the method for synthesizing these trialkylammonium salts by epoxidation reaction in a single step on the compounds of general formula (III):

CH₂═CH—R³⁰ ,X⁻ (III)

in which R represents a radical of general formula as defined above in (I),

by the action of oxidants of the peracid or peroxide type in aqueous or organic solution.

There may be mentioned, by way of example:

the peracids of general formula R′COOOH which are described in particular in Org. Reactions 1953,7,378, and preferably meta-chloroperoxybenzoic acid, alone or in combination with a base such as NaHCO₃, Na₂CO₃, K₂CO₃, or a phosphate,

the peroxides such as hydrogen peroxide H₂O₂, in the presence of a metal catalyst, such as the catalysts based on iron, molybdenum or manganese, and preferably tungsten in the form of acid, salts or oxides.

Another subject of the invention is the method for grafting trialkylammonium salts containing an epoxide functional group of formula I by reaction with the chemical functional groups containing labile hydrogens of the constituent macromolecules of the substrates intended to be subjected to grafting.

The functional groups containing labile hydrogens are the functional groups —OH, —SH, >NH, —CO(NH)— or —COOH, and the grafting level will be dependent on the level of functionalization of the substrate.

Among the substrates containing acid, alcohol, thiol, amide or amine functionalized branches, there may be mentioned, by way of example and without limitation, polysaccharide materials, polyamide materials, polyurethanes or polyimines, polyureas, phenol-formaldehyde resins, and all the materials produced by homopolymerization or copolymerization of monomers of the type such as acrylic or methacrylic acid or the ester derivatives thereof, vinylacetic acid and the ester derivatives thereof, butenedioic acid and the ester derivatives thereof, itaconic acid and the ester derivatives thereof, vinylsulfonic acid and the ester derivatives thereof, allyl alcohol and the ester derivatives thereof, esterified vinyl alcohol mild polyvinyl alcohols and the acrylamide or methacrylamide derivatives thereof, mercaptoethylene.

The expression polysaccharide materials is understood to mean the materials consisting of successions of several monosaccharides.

Most of them are natural, and the main one is cellulose and its derivatives, but also starch. There may be mentioned, by way of illustration, natural and plant fibers such as cotton, kapok, flax, hemp, ramie and jute, regenerated, modified or synthetic fibers such as viscose, rayon, meryl, zantrel and cellophane, which are used for textiles and packaging, but also wood, cardboard, paper used in furniture and in packaging.

The modified substrates according to the invention also have applications in cosmetology, in particular the cellulose matrices, and starch in the adhesive and paint industry.

The expression polyamide materials is understood to mean materials which include amide functional groups in the polymer chain, of which some are natural, for example polyamino acids or proteins.

Among the natural polyamides, there may be mentioned by way of illustration wool, silk and keratin.

Among the synthetic polyamides, there may be mentioned mainly the family of polyamides 6-6, for example Nylon®.

This method according to the invention comprises the steps of treating the substrate to be grafted, in a basic medium in an aqueous solution, then with a solution of trialkylammonium salt according to formula (I).

For example, for grafting onto cellulose, the reaction scheme is the following:

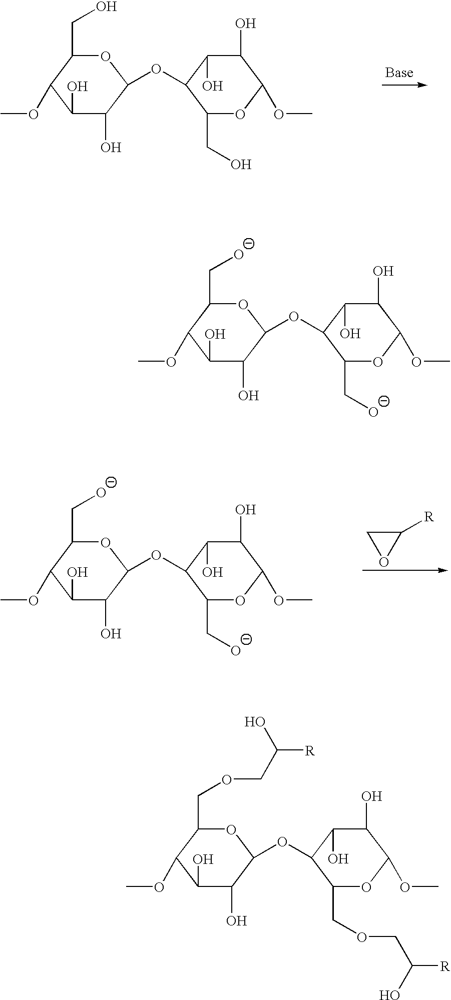

R represents a radical as defined in the general formula I.

The characteristics and subjects of the invention are demonstrated in the following examples:

### EXAMPLE 1

Preparation of a Basic Aqueous Solution of Epoxide at 25% by Mass

13.66 g of NaHCO₃ (0.163 mol) are added to 40 of an aqueous solution containing 50% by mass of undecylamidepropyltrimethylammonium methyl sulfate (CAS Registry Number: 94313-91-4) (0.051 mol). A solution of 14.02 (0.081 mol) of meta-chloroperbenzoic acid, dissolved in 530 ml of dichloromethane (concentration: 0.15 M) is then added dropwise (1.5 hours). The solution is maintained for 48 hours with vigorous stirring. After decantation and washing of the material with 40 ml of water, the aqueous phase is washed once with 100 ml of diethyl ether in order to remove the traces of meta-chlorobenzoic acid dissolved in the aqueous phase.

The solution obtained has a concentration by mass of 25%. It exhibits in proton NMR (D₂O) 18% of the starting alkene (multiplets at 5.8 and 4.95 ppm) and 82% of epoxide characterized by the multiplets at 3.7 ppm, 2.67 ppm and 2.92 ppm.

The biological tests of this basic aqueous solution of epoxide at 25% by mass gave an activity similar to that of the starting material at 50% in water.

### EXAMPLE 2

Preparation of a Neutral Aqueous Solution of Epoxide at 25% by Mass

A solution of 7.01 g (0.041 mol) of meta-chloroperbenzoic acid dissolved in 260 ml of dichloromethane (concentration: 0.16 M) is added dropwise (2.5 hours) to 20 g of an aqueous solution at 50% by mass of undecylamidepropyltrimethylammonium methyl sulfate (CAS Registry Number: 94313-91-4) (0.025 mol). The solution is maintained for 48 hours with vigorous stirring. After decantation and washing of the material with 20 ml of water, the aqueous phase is washed once with 100 ml of diethyl ether in order to remove the traces of meta-chlorobenzoic acid dissolved in the aqueous phase.

The solution obtained has a concentration by mass of 25%. It no longer exhibits in proton NMR (D₂O) traces of the starting alkene (multiplets at 5.8 and 4.95 ppm). The epoxidation therefore has a practically quantitative yield.

The epoxide is characterized in proton NMR in D₂O by the multiplets at 3.7 ppm, 2.67 ppm and 2.92 ppm.

### EXAMPLE 3

Preparation of a Neutral Aqueous Solution of Epoxide at 50% by Mass

A solution of 53.93 g (0.313 mol) of meta-chloroperbenzoic acid dissolved in 1 l of dichloromethane (concentration: 0.3 M) is added dropwise to 112 g of an aqueous solution at 50% by mass of undecylamidepropyltrimethylammonium methyl sulfate (CAS Registry Number: 94313-91-4) (0.198 mol). The solution is maintained for 48 hours with vigorous stirring. After decantation, the aqueous phase is washed once with 200 ml of diethyl ether in order to remove the traces of meta-chlorobenzoic acid dissolved in the aqueous phase.

The solution obtained no longer exhibits in proton NMR (D₂O) any trace of the starting alkene (multiplets at 5.8 and 4.95 ppm). The epoxidation therefore has a practically quantitative yield.

The epoxide is characterized in proton NMR in D₂O by the multiplets at 3.7 ppm, 2.67 ppm and 2.92 ppm.

The biological tests of this aqueous solution of epoxide at 50% by mass gave an activity similar to that of the starting material at 50% in water.

### EXAMPLE 4

Grafting of a Cotton Yarn with the Aid of a Basic Aqueous Solution of Epoxide at 25% by Mass

The cotton yarn is treated beforehand in aqueous sodium hydroxide at 1.5% by mass at boiling temperature for 3 hours. The yarn is then washed with water to neutrality, and then dried in an oven for 48 hours at 65° C.

1.986 g of dry treated cotton are placed in a bomb affording vertical mechanical stirring in 31.8 ml of isopropanol in the presence of 3.22 ml of aqueous sodium hydroxide at 15.8% by mass. The stirring is maintained at room temperature for 1 hour.

16.05 g of the basic aqueous solution of epoxide at 25% (example 1) and 2 ml of isopropanol are then added to the solution. The solution is stirred and heated at 65° C. for 5 hours.

After cooling to room temperature, the yarn is drained and then dried in an oven at 65° C. for 48 hours.

### EXAMPLE 5

Grafting of a Cotton Yarn with the Aid of a Neutral Aqueous Solution of Epoxide at 25% by Mass

The cotton yarn is treated beforehand in aqueous sodium hydroxide at 1.5% by mass at boiling temperature for 3 hours. The yarn is then washed with water to neutrality, and then dried in an oven for 48 hours at 65° C.

1.44 g of dry treated cotton are placed in a bomb affording vertical mechanical stirring in 23.1 ml of isopropanol in the presence of 2.34 ml of aqueous sodium hydroxide at 15.8% by mass. The stirring is maintained at room temperature for 1 hour.

11.64 g of the basic aqueous solution of epoxide at 25% (example 2) and 1.45 ml of isopropanol are then added to the solution. The solution is stirred and heated at 65° C. for 5 hours.

After cooling to room temperature, the yarn is drained and then dried in an oven at 65° C. for 48 hours.

### EXAMPLE 6

Grafting of a Cotton Yarn with the Aid of a Neutral Aqueous Solution of Epoxide at 50% by Mass

The cotton yarn is treated beforehand in aqueous sodium hydroxide at 1.5% by mass at boiling temperature for 3 hours. The yarn is then washed with wat r to neutrality, and then dried in an oven for 48 hours at 65° C.

1.477 g of dry treated cotton are placed in a bomb affording vertical mechanical stirring in 24 ml of isopropanol in the presence of 2.4 ml of aqueous sodium hydroxide at 15.8% by mass. The stirring is maintained at room temperature for 1 hour.

5.97 g of the basic aqueous solution of epoxide at 50% (example 3) and 1.5 ml of isopropanol are then added to the solution. The solution is stirred and heated at 65° C. for 5 hours.

After cooling to room temperature, the yarn is drained and then dried in an oven at 65° C. for 48 hours.

### EXAMPLE 7

Grafting of a Cotton Yarn with the Aid of a Neutral Epoxide Solution at 142.5 g/l in Isopropanol

The cotton yarn is treated beforehand in aqueous sodium hydroxide at 1.5% by mass at boiling temperature for 3 hours. The yarn is then washed with water to neutrality, and then dried in an oven for 48 hours at 65° C.

1.038 g of dry treated cotton are placed in a bomb affording vertical mechanical stirring in 17 ml of isopropanol in the presence of 1.7 ml of aqueous sodium hydroxide at 15.8% by mass. The stirring is maintained at room temperature for 1 hour.

14.8 g of a neutral solution of evaporated epoxide (example 3) diluted to 142.5 g/l in isopropanol are then added to the solution. The solution is stirred and heated at 65° C. for 5 hours.

After cooling to room temperature, the yarn is drained and then dried in an oven at 65° C. for 48 hours.

### EXAMPLE 8

Grafting of a Cotton Yarn with the Aid of a Neutral Aqueous Solution of Epoxide at 50% by Mass in the Presence of 1,2-propanediol (MPG)

The cotton yarn is treated beforehand in aqueous sodium hydroxide at 1.5% by mass at boiling temperature for 3 hours. The yarn is then washed with water to neutrality, and then dried in an oven for 48 hours at 65° C.

1.753 g of dry treated cotton are placed in a bomb affording vertical mechanical stirring in 28 ml of 1,2-propanediol (MPG) in the presence of 2.8 ml of aqueous sodium hydroxide at 15.8% by mass. The stirring is maintained at room temperature for 1 hour.

7.08 g of an aqueous solution of epoxide at 50% (example 3) and 1.8 ml of MPG are then added to the solution. The solution is stirred and heated at 65° C. for 5 hours.

After cooling to room temperature, the yarn is drained and then dried in an oven at 65° C. for 48 hours.

### EXAMPLE 9

Grafting of a White Knitted Cotton Fabric with the Aid of a Neutral Aqueous Solution of Epoxide at 50% by Mass

1.104 g of white knitted cotton fabric are placed in a bomb affording vertical mechanical stirring in 18 ml of isopropanol in the presence of 1.8 ml of aqueous sodium hydroxide at 15.8% by mass. The stirring is maintained at room temperature for 1 hour.

4.47 g of an aqueous solution of epoxide at 50% (example 3) and 1.2 ml of isopropanol are then added to the solution. The solution is stirred and heated at 65° C. for 5 hours.

After cooling to room temperature, the knitted fabric is drained and then dried in an oven at 65° C. for 48 hours. The knitted fabric obtained shows a contact bactericidal effect according to the two bacteriological tests specific to knitted fabrics, described in example 15.

### EXAMPLE 10

Grafting of a Raw Knitted Cotton Fabric with the Aid of a Neutral Aqueous Solution of Epoxide at 50% by Mass

The raw knitted cotton fabric is treated beforehand in aqueous sodium hydroxide at 1.5% by mass at boiling temperature for 3 hours. The knitted fabric is then washed with water to neutrality, and then dried in an oven for 48 hours at 65° C.

1.157 g of dry treated knitted cotton fabric are placed in a bomb affording vertical mechanical stirring in 19 ml of isopropanol in the presence of 1.9 ml of aqueous sodium hydroxide at 15.8% by mass. The stirring is maintained at room temperature for 1 hour.

4.67 g of an aqueous solution of epoxide at 50% (example 3) and 1.2 ml of isopropanol are then added to the solution. The solution is stirred and heated at 65° C. for 5 hours.

After cooling to room temperature, the knitted fabric is drained and then dried in an oven at 65° C. for 48 hours. The knitted fabric obtained shows a contact bactericidal effect according to the two bacteriological tests specific to knitted fabrics, described in example 15.

### EXAMPLE 11

Grafting of a Viscose Yarn with the Aid of a Neutral Aqueous Solution at 50% by Mass

1.420 g of dry treated viscose yarn are placed in a bomb affording vertical mechanical stirring in 23 ml of isopropanol in the presence of 2.3 ml of aqueous sodium hydroxide at 15.8% by mass.

The stirring is maintained at room temperature for 1 hour.

5.74 g of an aqueous solution of epoxide at 50% (example 3) and 1.5 ml of isopropanol are then added to the solution. The solution is stirred and heated at 65° C. for 5 hours.

After cooling to room temperature, the yarn is drained and then dried in an oven at 65° C. for 48 hours.

### EXAMPLE 12

Grafting of a Natural Woolen Yarn with the Aid of a Neutral Aqueous Solution of Epoxide at 50% by Mass without Prior Basic Treatment.

1.618 g of natural woolen yarn are placed in a bomb affording vertical mechanical stirring in 26 ml. The stirring is maintained at room temperature for 1 hour.

6.472 g of an aqueous solution of epoxide at 50% (example 3) and 1.6 ml of isopropanol are then added to the solution. The solution is stirred and heated at 40° C. for 5 hours.

After cooling to room temperature, the yarn is drained and then dried in an oven at 65° C. for 48 hours.

### EXAMPLE 13

Grafting of a Natural Woolen Yarn with the Aid of a Neutral Aqueous Solution of Epoxide at 50% by Mass with Prior Basic Treatment

1.856 g of natural woolen yarn are placed in a bomb affording vertical mechanical stirring in 30 ml with 3 ml of an aqueous solution of potassium carbonate (K₂CO₃) at 10% by mass. The stirring is maintained at room temperature for 1 hour.

7.424 g of an aqueous solution of epoxide at 50% (example 3) and 1.9 ml of isopropanol are then added to the solution. The solution is stirred and heated at 40° C. for 5 hours.

After cooling to room temperature, the yarn is drained and then dried in an oven at 65° C. for 48 hours.

### EXAMPLE 14

Results and Tests of Biological Activity

The principle of characterization is the deposition of a bacterial suspension on a piece of sample to be analyzed and the counting of viable bacteria after a previously established contact time, in comparison with a nontreated control.

The strain used is Staphylococcus aureus.

The sample to be tested is placed in a sterile flask and then 0.4 ml of bacterial suspension at 4.5×10⁵ bacteria/cm³ is deposited on this sample.

The contact is maintained at 30±1° C. for 24 hours between the sample and the bacterial solution.

After the contact time has elapsed, 10 ml of diluent (tryptine: 1 g; NaCl: 8.5 g; water: qs 1 l) are poured into the flask, with vigorous stirring, in order to detach the bacteria from the substrate.

Counting of the viable bacteria is then carried out by the method of the most probable number (MPN) of bacteria.

This operation should be performed on four identical samples in the presence of a control containing no biocidal molecule.

Let Nₜ be the mean number of colonies counted, let N₀ be the mean number of colonies deposited on the sample at time T₀ (4.5×10⁵ bacteria/cm³), the activity is defined by the logarithm of the ratio Nₜ/N₀ (logarithmic reduction).

The results obtained on four samples before washing and after washing are assembled in table 1.

There will be noted the permanence of the activity, in particular for sample No. 4, for which after 20 washes the activity undergoes no alteration.

TABLE I

| | Washes | N_{T} | Log (N_{T}/N_{O}) |
|---|---|---|---|
| Example 4 | 0 | <10³ | <−2.65 |
| | 3 | 9.5 × 10³ | −1.68 |
| | 6 | 4.5 × 10⁴ | −1.00 |
| | 9 | 4.5 × 10⁵ | 0 |
| | 12 | 2.5 × 10⁶ | 0.76 |
| Example 5 | 0 | <10³ | <−2.65 |
| | 3 | <10³ | <−2.65 |
| Example 6 | 0 | <10³ | <−2.65 |
| | 3 | <10³ | <−2.65 |
| | 0 | <10³ | <−2.65 |
| | 6 | <10³ | <−2.65 |
| | 9 | <10³ | <−2.65 |
| | 20 | <10³ | <−2.65 |
| Example 7 | 0 | <10³ | <−2.65 |
| | 3* | <2.5 × 10³ | <−2.65 |
| | 9 | <2.5 × 10³ | <−2.65 |
| Example 8 | 3 | <10³ | <−2.65 |
| | 20 | <10³ | <−2.65 |
| Example 11 | 6 | <10³ | <−2.65 |
| | 6 | | −2.65 |
| Example 12 | 3 | <10³ | <−2.65 |
| Example 13 | 3 | <10³ | <−2.65 |

*contact time: 5 days

### EXAMPLE 15

Tests of Biological Activity for the Knitted Fabrics

The tests are carried out with the bacterium Staphylococcus aureus in a Petri dish. Two sorts of bacteriological tests were used. Disks 3 cm in diameter are cut out in the surface of the textile to be tested. The test is to be carried out on three test pieces. To avoid contaminations on the agar (various fungi and bacteria), the samples are sterilized.

Test 1:

The test piece to be tested (control or treated) is placed on nutrient agar inoculated with the appropriate test bacteria (0.1 ml at 10⁵ bact/ml) in a Petri dish.

Treated test piece: the antibacterial effect of the treatment manifests itself after incubation at 30° C. by the fact that the zone of the nutrient agar plate which was in contact with the test piece (zone of contact) is free of bacteria. It is sometimes possible to observe an additional zone with no development of bacteria around the edge of the test piece. That is the inhibitory zone which denotes some departure of the antiseptic. It demonstrates the “nonsolidity” of the attachment of the biocide in the fiber.

Control test piece: a considerable bacterial invasion under the control test piece, with no inhibitory zone, is observed.

Test 2:

0.1 ml of culture broth containing about 10⁵ bact/ml is deposited on the test piece to be tested (control or treated), which is itself on a sterile empty Petri dish. Care should be taken for good penetration of the drop into the test piece: it is sometimes found to be necessary to add a wetting agent to the culture broth.

The test piece is immediately deposited on the sterile agar (t=0 h), the Petri dish is then placed in an incubator at 30° C. for 24 hours.

It is also possible to wait for 24 hours before depositing the test piece on the sterile agar (t=24 h). The incubation is continued for 24 hours in an incubator at 30° C.

The antibacterial effect is manifested, after this period of incubation, by the absence of bacterial development under the treated test piece. A considerable bacterial development is observed under the control.

### EXAMPLE 16

Protocol for Washing the Samples in Order to Mimic the Action of Washing

100 ml of demineralized water are added to about 1.5 g of fiber (or of knitted fabric) placed in an apparatus affording vertical stirring.

The stirring is maintained for 5 minutes and then the fiber (or the knitted fabric) is drained.

### EXAMPLE 17

Grafting of a Cotton Fabric in an Industrial Machine, with the Aid of a Neutral Aqueous Solution of Epoxide at 50% by Mass.

For subsequent development, grafting onto a white cotton fabric was carried out on a Jigger type pilot machine. The results obtained on this type of machine are directly transportable to the industrial level.

The grafted fabric is a band of white cotton 0.25 m in width by 6 m in length, that is a total surface area of 1.25 m². The fabric weighs 162.5 g per m², the treatment is therefore carried out with a bath volume of 3 L according to the protocol below.

The bath is filled with 0.3 L of aqueous sodium hydroxide at 16% by mass and 2.7 L of 1,2-propanediol. The fabric is circulated at medium speed for 1 hour.

The bath is emptied and then filled with 650 g of epoxide at 50% in water and 2.3 L of 1,2-propanediol. The fabric is circulated at medium speed at the same time as the heating (T=0). The desired temperature (65° C.) is reached in 10 minutes and then maintained for 4 hours.

The samples collected at T=10 min, 1h10′, 2h40′ and 4h10 include the portion of fabric which was not immersed (between the bath and the roll).

The samples collected at 1h10′, 2h40′ and 4h10 show a contact biocidal activity. The treatment is therefore effective from 1 h.

The absence of an inhibitory zone around the sample makes it possible to visualize the absence of release of the active ingredient.

1. An epoxidized trialkylammonium salt, characterized in that it is chosen from the salts of general formula (I): 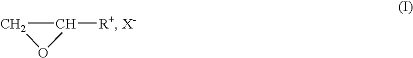
in which:
R represents a group:
—(CH₂)ₙ—A—NH—(CH₂)_{n′}—N⁺R₁R₂R₃
in which:
A represents a group —CO— or —CH₂—
n′ and n are each between 1 and 20, with (n+n′) greater than or equal to 10,
R₁, R₂ and R₃ are linear or branched C₁ to C₂₀ alkyl or monohydroxyalkyl groups, which may be identical or different,
X⁻ represents the counter-ion.

2. The epoxidized trialkylammonium salt as claimed in claim 1, characterized in that —R represents the radical:
—(CH₂)₈—CO—NH—(CH₂)₃—N⁺(CH₃)₃

3. A method for synthesizing epoxidized trialkylammonium salts as claimed in claim 1 or 2, characterized in that it consists in reacting a compound of general formula CH2═CH—R⁺,X⁻ in which R r presents a group:
—(CH₂)ₙ—A—NH—(Ch₂)_{n′}—N⁺R₁R₂R₃
in which:
A represents a group —CO— or —CH₂—
n′ and n are between 1 and 20, with (n+n′) greater than or equal to 10,
R₁, R₂ and R₃ are linear or branched C1 to C4 alkyl or monohydroxyalkyl groups, which may be identical or different.
X⁻ represents the counter-ion.
With an oxidant of the peracid or peroxide type in aqueous or organic solution.

4. A method for covalent grafting of the trialkylammonium salts as claimed in claim 1 or 2, characterized in that it comprises the steps of treating a substrate to be grafted in a basic medium in an aqueous or organic solution, and then treating with a solution of trialkylammonium salts as claimed in claim 1 or 2.

5. The method as claimed in claim 4, characterized in that the macromolecules of the substrate contain labile hydrogens.

6. A biocidal substrate which can be obtained by a method of grafting as claimed in claim 4.

7. The use of the trialkylammonium salts as claimed in claims 1 and 2 for obtaining a substrate which is biocidal or bacteriostatic by contact.
